(19) United States
(12) Patent Application Publication
(10) Pub. No.: US 2010/0179462 A1
(43) Pub. Date: Jul. 15, 2010

(54) Stabilizing Brace For Use By Individuals Having Neuromuscular Disorders

(76) Inventor: Valentino Costanzo, Chittenango, NY (US)

Correspondence Address: BOND, SCHOENECK & KING, PLLC, ONE LINCOLN CENTER, SYRACUSE, NY, 13202-1355, US

(21) Appl. No.: 12/353,389

(22) Filed: Jan. 14, 2009

(51) Int. Cl. A61F 5/00

(52) U.S. Cl. 602/20

(57) ABSTRACT

A brace for supporting the arms or wrists of a user afflicted with a muscular disorder is provided. The brace generally includes a yoke section having first and second terminal ends and being adapted for positioning over the shoulders and behind the neck of the user, and a pair of opposed arm/wrist supports that extend outwardly from respective ones of the first and second terminal ends of the yoke portion and being adapted to support the arms/wrists of the user.

### BACKGROUND OF THE INVENTION

The present invention relates generally to medical braces, and more particularly to braces that aid a person suffering from muscular disorders that are characterized by uncontrollable shaking and/or tremors of the hands.

Muscular disorders characterized by muscle rigidity, tremor, a slowing of physical movement (bradykinesia) and, in extreme cases, a loss of physical movement (akinesia) afflict a large number of people, both young and old alike. Parkinson's Disease is a common form of muscular disorder a symptom of which can be the uncontrollable shaking or tremoring of a person's hands.

The shaking or tremors that afflict many sufferers of muscular disorders, such as Parkinson's, can sometimes be controlled if the arm or wrist of the person is braced in some manner to counter the forces that otherwise cause the person's hands to move uncontrollably. For example, the handwriting of a person afflicted with a muscular disorder such as Parkinson's can become unintelligible as a consequence of the shakiness to which the person's writing strokes are subject. If the person's writing hand is braced in some manner, however, such as by resting it on the table surface or held with some form of brace, the legibility of the handwriting may improve.

The degree to which a person afflicted with a muscular disorder characterized at least in part by shaky hands can perform such routine grooming activites as shaving, brushing his or her teeth, or brushing his or her hair can vary widely. In degenerative cases, the severity of the shaking may prevent the person from being able to perform these typical grooming activites due to the lack of bracing of the hands available when having to raise one's hands to face level. In such dire circumstances, the individual needs to either find alternative grooming sources, such as using an electric razor instead of a fixed blade razor which could cause injury due to the shaking of the razor near one's face or head, or have a caretaker's assistance in performing these personal grooming tasks.

### SUMMARY OF THE INVENTION

It is therefore an object and advantage of the present invention to provide a brace that will support a user's hands when raised to face level.

It is another object and advantage of the present invention to provide a brace to assist a person afflicted with a muscular disorder in performing personal grooming of one's face and hair.

It is another object and advantage to provide a brace that is simple to manipulate by its user.

It is another object and advantage of the present invention to provide a brace that can be adjusted to be used with differently sized users.

Other objects and advantages of the present invention will in part be obvious, and in [part appear hereinafter.

In accordance with the foregoing objects and advantages, brace for supporting the arms or wrists of a user afflicted with a muscular disorder is provided. In one aspect of the invention the brace generally comprises a yoke section having first and second terminal ends and being adapted for positioning over the shoulders and behind the neck of the user, and a pair of opposed arm/wrist supports that extend outwardly from respective ones of the first and second terminal ends of the yoke portion and being adapted to support the arms/wrists of the user. In a separate aspect of the invention, the lengths and/or widths of the yoke portion and its legs, and the wrist/arm supports can be adjusted via telescoping sections that are fixed in place via spring biased pins with corresponding openings, or like fastening mechanisms.

In another aspect of the present invention, a method for a user afflicted with a muscular disorder to perform self grooming tasks in the vicinity of the user's face or head is provided. The method generally comprises the steps of providing a brace having a yoke portion having first and second terminal ends and being adapted for positioning over the shoulders and behind the neck of the user and a pair of opposed arm/wrist supports that extend outwardly from respective ones of the first and second terminal ends of the yoke portion and being adapted to support the arms/wrists of the user, placing the yoke portion of the brace over the user's shoulders and behind the user's neck such that the opposing arm/wrist supports that extend outwardly from the yoke portion extend forwardly from the user's chest region, and positioning at least one of the user's arms/wrists on at least one respective arm/wrist support that extends outwardly from the yoke portion.

### BRIEF DESCRIPTION OF THE DRAWINGS

The present invention will be more fully appreciated and understood by reading the following Detailed Description in conjunction with the accompanying drawings, in which:

FIG. 1 is a perspective view illustrating the present invention being worn by a user;

FIG. 2 is a perspective view of the brace; and

FIG. 3 is a perspective view showing an alternate embodiment of the brace.

### DETAILED DESCRIPTION

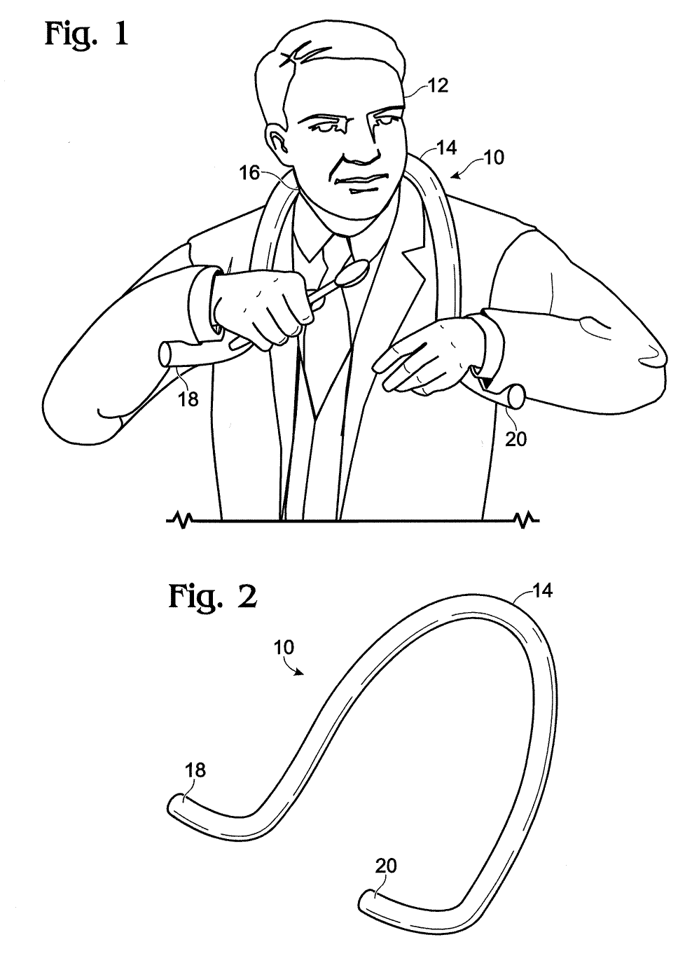

With reference to the drawings, wherein like reference numerals refer to like parts throughout, there is seen in FIG. 1 a brace designated generally by reference numeral 10, for use by an individual 12 afflicted with a muscular disorder, such as Parkinson's Disease. Brace 10 includes a yoke portion 14 adapted to be supported around the user's neck 16, and a pair of arm or wrist support legs 18, 20 that extend outwardly and slightly upwardly from the ends of the yoke portion 14. Support legs 18, 20 are adapted to support and stabilize the user's arm or wrist 22 when the user is performing grooming tasks such as brushing his or her teeth, combing his or her hair, shaving his face, and like activities.

Yoke portion 14 is generally shaped such that it be loosely placed over the shoulders and behind the neck 16 of user 10, and its legs are shaped to contour the typical user's upper body shape. Legs 18, 20 are preferably integral with yoke portion 14, although they could be attached as separate members using fasteners such as cotter pins, spring biased pins that engage openings (as shown in FIG. 1) to provide adjustability, or the like. Brace 10 is preferably composed of a semi-rigid materials and coated with a foam or other soft or pliable material that will be comfortable for the user.

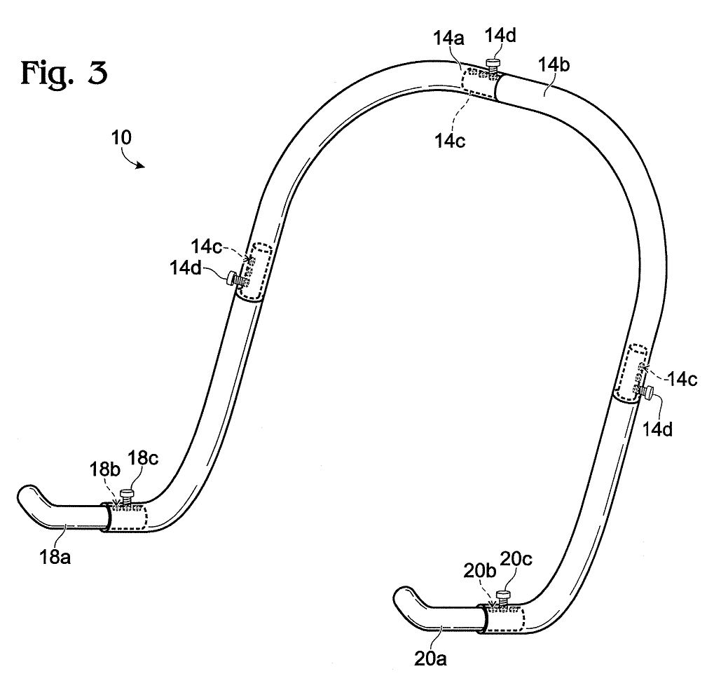

With respect to FIG. 2, the legs and neck support region of yoke 14 as well as the arm/wrist supports are contemplated as being adjustable via telescoping sections 14a, 14b, 18a, 20a, respectively. The adjustment can be achieved through the use of fixed openings 14c, 18b, 20b, that engage spring biased pins 14d, 18c, 20c, or similar fastening mechanisms.

What is claimed is:

1. A brace for supporting the arms or writs of a user afflicted with a muscular disorder, comprising:
a. a yoke section having first and second terminal ends and being adapted for positioning over the shoulders and behind the neck of the user; and
b. a pair of opposed arm/wrist supports that extend outwardly from respective ones of said first and second terminal ends of said yoke portion and being adapted to support the arms/wrists of the user.

2. The brace according to claim 1, wherein said yoke section and said pair of opposed arm/wrist supports comprise exterior surfaces that are at least partially composed of a foam material.

3. The brace according to claim 1, wherein said yoke portion is of an adjustable width and includes first and second legs that are of adjustable lengths.

4. The brace according to claim 3, wherein said pair of arm/wrist are adjustably connected to said yoke portion.

5. The brace according to claim 1, wherein said pair of arm/wrist are adjustably connected to said yoke portion

6. A method for a user afflicted with a muscular disorder to perform self grooming tasks in the vicinity of the user's head, comprising the steps of:
a. providing a brace having a yoke portion having first and second terminal ends and being adapted for positioning over the shoulders and behind the neck of the user and a pair of opposed arm/wrist supports that extend outwardly from respective ones of said first and second terminal ends of said yoke portion and being adapted to support the arms/wrists of the user;
b. placing the yoke portion of the brace over the user's shoulders and behind the user's neck such that the opposing arm/wrist supports that extend outwardly from the yoke portion extend forwardly from the user's chest region; and
c. positioning at least one of the user's arms/wrists on at least one respective arm/wrist support that extends outwardly from the yoke portion.

7. The method according to claim 6, wherein the step of positioning at least one of the user's arms/wrists on at least one respective arm/wrist support that extends outwardly from the yoke portion includes the user placing both of his or her arms/wrists in respective ones of the wrist/arm supports the extend outwardly from the yoke portion.

8. The method according to claim 6, comprising the further step of adjusting the width of the yoke portion.

9. The method according to claim 6, comprising the further step of adjusting the length of the arm/wrist supports.
